(19) United States
(12) Patent Application Publication
(10) Pub. No.: US 2007/0054332 A1
(43) Pub. Date: Mar. 8, 2007

(54) Syndecan 1 ectodomain inhibits cancer

(76) Inventors: Alan Rapraeger, Madison, WI (US); Yan Ji, Madison, WI (US)

Correspondence Address: FULBRIGHT & JAWORSKI L.L.P., 600 CONGRESS AVE., SUITE 2400, AUSTIN, TX, 78701, US

(21) Appl. No.: 11/501,209

(22) Filed: Aug. 8, 2006

Related U.S. Application Data
(60) Provisional application No. 60/707,020, filed on Aug. 10, 2005.

(51) Int. Cl. G01N 33/574; A61K 38/17; A61K 38/10; A61K 38/08

(52) U.S. Cl. 435/7.23; 514/12; 514/13; 514/14; 514/15; 514/16; 514/17

(57) ABSTRACT

The present invention provides for the treatment of cancers, e.g., carcinomas, using the ectodomain of syndecan 1, where the ectodomain lacks heparan sulfate residues. In addition, agents that bind the ectodomain of syndecan 1 also are useful as anti-cancer therapeutics.

This application claims benefit of priority to U.S. Provisional Application Ser. No. 60/707,020, filed Aug. 10, 2005, the entire contents of which are hereby incorporated by reference.

The government owns rights in the present invention pursuant to grant number CA 109010 from the National Institutes of Health.

### BACKGROUND OF THE INVENTION

1. Field of the Invention

The present invention relates to fields of molecular biology and oncology. More particularly, the present invention relates to use of the ectodomain of syndecan 1 in the treatment of cancer.

2. Description of Related Art

The syndecans are a four-member family of transmembrane cell surface PGs that bear heparan sulfate glycosaminoglycan (GAG) chains. The syndecans are expressed on virtually all cell types throughout development and adulthood, and their expression can be altered under certain pathophysiological conditions, including the processes of tumor onset, progression and metastasis (Sanderson, 2001; Sasiskeharan et al., 2002). Their heparan sulfate chains endow these receptors with the ability to bind numerous “heparin”-binding growth factors and morphogens, for which heparan sulfate is an important regulator. The heparan sulfate chains also bind to “heparin”-binding sites present in matrix ligands, including fibronectin, vitronectin, laminins and the fibrillar collagens (Bernfield et al., 1999). As such, the syndecans are believed to have roles in cell adhesion and signaling, possibly as co-receptors with integrins and cell-cell adhesion molecules.

The syndecan core proteins share a high degree of conservation in their short (ca. 30 amino acids) cytoplasmic domains and transmembrane domains; in contrast, the extracellular domains (also known as ectodomains) are divergent with the exception of consensus sites for GAG attachment. In the highly conserved cytoplasmic domains, a juxtamembrane C1 region is exactly conserved (with the exception of a conservative R for K amino acid substitution in syndecan-3) among the syndecan across all species and has been implicated in binding protein 4.1, ezrin, radixin and moesin (FERM) domains (Cohen et al., 1998; Rapraeger and Ott, 1998; Hsueh et al., 2001; Granes et al., 2000). A C-terminal C2 region consisting of the amino acid sequence EFYA is present in all syndecans and binds to post-synaptic density 95, PSD-95; discs large, Dlg; zonula occludens-1, ZO-1 (PDZ) domains in several proteins including calcium/calmodulin-dependent serine protein kinase (CASK) (Hsueh et al., 1998), syntenin (Grootjans et al., 1997), synbindin (Ethell et al., 2000) and synectin (Gao et al., 2000). The intervening variable (V) region is distinct for each of the four family members, but its syndecan-specific identity is conserved across species. The function of this domain is largely unknown.

Syndecan-deficient Raji lymphoblastoid cells when transfected to express Sdc1 acquire the ability to bind and spread on Sdc1 antibody or matrix ligands (Lebakken and Rapraeger, 1996; Lebakken et al., 2000). This signaling depends on the transmembrane domain of the PG, which is found in specialized lipid domains, as well as a region within the Sdc1 extracellular domain (McQuade and Rapraeger, 2003). This unique finding suggests that the syndecan extracellular protein domains have important functions independent of, but most likely supplemented by, their attached GAG chains. Transfection of Sdc1 in COS-7 cells also stimulates cell spreading, although the phenotype in these cells is characterized by fascin microspike formation and membrane ruffling (Adams et al., 2001). Transfection studies with various Sdc1 mutants mapped the active domain required for cell spreading to the ectodomain.

A role for syndecan 1 in cancer has been advanced. Mali et al. (1994) reported that free ectodomain from the culture medium of syndecan 1-transfected S115 mouse mammary tumor cells or normal murine mammary gland cells can suppress the growth of S115 tumor cells at nanomolar concentrations. Intact heparan sulfate structure of the ectodomain was required for the suppression as degradation of heparan sulfate chains completely abolished tumor cell growth inhibition. However, this observation has not been followed further.

### SUMMARY OF THE INVENTION

Thus, in accordance with the present invention, there is provided a method of inhibiting a cancer cell comprising contacting said cancer cell with a syndecan 1 ectodomain peptide, wherein said peptide lacks heparan sulfate residues. The peptide may be between 5 and 100 residues in length, between 15 and 50 residues in length or between 25 and 40 residues in length. Specific lengths include 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 residues. The cancer cell may be an epithelial cancer cell or a carcinoma. The cancer cell may also be a prostate cancer cell, a lung cancer cell, a stomach cancer cell, a brain cancer cell, a breast cancer cell, an ovarian cancer cell, a skin cancer cell, a colon cancer cell, a cervical cancer cell, a liver cancer cell, a head & neck cancer cell, an esophageal cancer cell, a pancreatic cancer cell, a testicular cancer cell, or a blood cancer cell. The cancer cell may be a drug-resistant cancer cell or metastatic cancer cell. Inhibiting may comprise one or more of inhibiting cancer cell growth, inhibiting cancer cell proliferation, or inhibiting cancer cell differentiation. The method may further comprise contacting said cancer cell with a second anti-cancer treatment, for example, radiation, chemotherapy, hormonal therapy, gene therapy or immunotherapy. The method may also further comprise contacting said cancer cell a second time with said peptide.

In another embodiment, there is provided a method of treating a subject with cancer comprising administering to said subject a syndecan 1 ectodomain peptide, wherein said peptide lacks heparan sulfate residues. The peptide may be between 5 and 100 residues in length, between 15 and 50 residues in length or between 25 and 40 residues in length. Specific lengths include 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 residues. The cancer cell may be an epithelial cancer cell or a carcinoma, a prostate cancer cell, a lung cancer cell, a stomach cancer cell, a brain cancer cell, a breast cancer cell, an ovarian cancer cell, a skin cancer cell, a colon cancer cell, a cervical cancer cell, a liver cancer cell, a head & neck cancer cell, an esophageal cancer cell, a pancreatic cancer cell, a testicular cancer cell, or a blood cancer cell. The cancer cell may be a drug-resistant cancer cell or metastatic cancer cell. Inhibiting may comprise one or more of inhibiting cancer cell growth, inhibiting cancer cell proliferation, or inhibiting cancer cell differentiation. The method may further comprise contacting said cancer cell with a second anti-cancer treatment, for example, radiation, chemotherapy, hormonal therapy, gene therapy or immunotherapy. The method may also further comprise contacting said cancer cell a second time with said peptide.

In yet another embodiment, there is provided a method of treating a subject with cancer comprising contacting said cancer cell with an agent that binds syndecan 1. The agent may be a peptide, peptidomimetic or polypeptide, such as an antibody or antibody fragment, or an organopharmaceutical. The cancer may be an epithelial cancer cell or a carcinoma, a prostate cancer, a lung cancer, a stomach cancer, a brain cancer, a breast cancer, an ovarian cancer, a skin cancer, a colon cancer, a cervical cancer, a liver cancer, a head & neck cancer, an esophageal cancer, a pancreatic cancer, a testicular cancer, or a blood cancer. Inhibiting may comprise one or more of inhibiting cancer cell growth, inhibiting cancer cell proliferation, inhibiting cancer cell differentiation, inhibiting cancer progression, inhibiting cancer metastasis, or inhibiting cancer recurrence. The cancer cell may be a drug-resistant cancer cell or a metastatic cancer cell. The method may further comprise contacting said cancer cell with a second anti-cancer treatment, for example, radiation, chemotherapy, hormonal therapy, gene therapy or immunotherapy. The method may also further comprise contacting said cancer cell a second time with said peptide.

In still yet another embodiment, there is provided a method of screening for a candidate anti-cancer agent comprising (a) providing a syndecan 1 ectodomain; (b) contacting said syndecan 1 ectodomain with test compound; (c) assessing binding of said test compound to said syndecan 1 ectodomain; wherein an agent that binds to said syndecan 1 ectodomain is a candidate anti-cancer agent. The syndecan 1 ectodomain may be comprised within full length syndecan 1. The full length syndecan 1 may be embedded in a membrane, such as in an intact cell membrane, such as that from a cell recombinantly engineered to express syndecan 1. Alternatively, the syndecan 1 ectodomain may be fixed to a support, such as a stick, a well, a bead or a column matrix. Binding may be detected by mass spectrometry, cell sorting, fluorescence, sedimentation, electrical current, FRET, chromatography, and solid phase adhesion. The method may also further comprise assessing the effect of said test compound on cancer cell growth. The test compound may be a peptide, a polypeptide, a peptidomimetic, a nucleic acid, a carbohydrate, a lipid, or a organopharmaceutical.

A further embodiment comprises a method of screening a syndecan 1 ectodomain peptide for anti-cancer activity comprising (a) providing a syndecan 1 ectodomain peptide; (b) contacting said peptide with a cancer cell; and (c) assessing the effect of said peptide on said cancer cell growth or viability, wherein a peptide that inhibits cancer cell growth or viability has anti-cancer activity.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Syndecan-1-specific polyclonal antibody (a-S1ED-pAb) blocks the accumulation of human mammary epithelial cells in matrigel. Normal MCF10A, tumorigenic MCF-7 and highly invasive MDA-MB-231 human mammary epithelial cells were plated in 10% Matrigel™ containing either no treatment, or 200 μg/ml of a nonspecific control antibody (anti-GST) or a specific rabbit polyclonal antibody preparation specific for the extracellular domain of Sdc1 (anti-S1ED) for 6 days. They were compared with the mouse NMuMG mammary epithelial cell line. Cells were viewed either in phase, or with fluorescent Hoechst staining to mark nuclei. The untreated MCF10A cells proliferate and differentiate to form hollow acini, but fewer and only single cells are seen in the presence of the Sdc1 antibody. The untreated MCF-7 cells form cell-filled acini, but treatment with the syndecan antibody prevents cell accumulation. Untreated MDA-MB-231 cells proliferate to form invasive sheets or cords of cells, but reduced numbers are seen and only as single cells in the presence of anti-S1ED.

FIG. 2—Proliferation of human mammary carcinoma cell lines in 3D matrigel. Cells are plated in matrigel for up to 6 days. The matrigel is then dissolved and the total cell number is determined by cell counting. The cells are either exposed to no treatment (NT), 200 μg/ml nonspecific rabbit IgG (IgG) or 200 μg/ml antibody raised against the mouse Sdc1 ectodomain (anti-mS1ED).

FIGS. 3A-B—Silencing expression of syndecan-1 causes cell cycle arrest of mamamary carcinoma cells. MDA-MB-231 human mammary carcinoma cells are transfected using lipofectamine with siRNA oligos that specifically silence expression of human Sdc1 (siRNA) and are plated in matrigel for three days. The cells are then released from the matrigel and stained with Hoescht dye, specific for DNA, or with mAb B-B4, specific for human Sdc1. The cells were then subjected to flow cytometry to quantify DNA and Sdc1 (FIG. 3A). Silencing of Sdc1 expression with siRNA mimicks the block to cell proliferation seen during treatment with Sdc1 antibody, and arrests cells in the G₀/G₁ phase of the cell cycle. Note that the cells expressing diminished amounts of Sdc1 are clustered in the G₀/G₁ phase. The percentage of cells in G2, M or G₀/G₁ of the cell cycle phase is quantified in (FIG. 3B).

FIG. 4—MCF7 human mammary carcinoma and the HaCat human keratinocyte cell lines display reduced cell growth when Sdc1 expression is silenced. MCF7 (A) and HaCat (B) cells were transfected using lipofectamine with siRNA oligos specific for human Sdc1 and allowed to recover for 24 hr. The growth of the cells over the subsequent 72 hr is shown, comparing the cells treated with the siRNA (RNAi) with the cells treated with lipofectamine alone (Control).

FIG. 5—Expression of the syndecan-1 extracellular domain at the cell surface is sufficient to regulate cell proliferation. MDA-MB-231 cells are transfected using lipofectamine with siRNA oligos that specifically silence expression of human Sdc1 (siRNA). In addition, cells are transfected with cDNA encoding the extracellular domain of mouse Sdc1 linked to the plasma membrane by a glycosylphosphatidylinositol (GPI) linkage (siRNA+GPI-mS1ED). Expression of this mouse chimera is not affected by the siRNA and its expression is sufficient to rescue the block to cell proliferation seen with siRNA treatment alone. The cells are plated in matrigel for three days, after which time the efficacy of the siRNA begins to wane. The cells are stained with DAPI, a fluorescent DNA stain, to visualize the nuclei and cell numbers (quantified in the bar graph). Colonies of cells that have arisen via cell proliferation are outlined in dashed circles.

FIG. 6.—Sdc1 mutant lacking heparan sulfate chains can regulate cell proliferation. Human Sdc1 expression was silenced using siRNA oligos either in parental MDA-MB-231 cells expressing a control vector (NEO), or cells expressing native full-length mouse Sdc1 (FL) or a mouse Sdc1 mutant lacking its heparan sulfate chains (TDM mutant). Cells were allowed to recover for 24 hr, then their growth was quantified over the subsquent 48 hr. Note that proliferation is rescued by either the native mouse Sdc1 (FL) or by the Sdc1 lacking its heparan sulfate chains (TDM).

### DESCRIPTION OF ILLUSTRATIVE EMBODIMENTS

The syndecans, a family of four cell surface receptors (heparan sulfate proteoglycans), are cell adhesion receptors and regulators of signaling by growth factors/growth factor receptors and cell adhesion receptors, especially integrins. They are expressed on all adherent cells and regulate cell behavior during development and cancer. The present inventors have focused on Sdc1, which is abundant on epithelial cells, and appears necessary for the maintenance of normal epithelial cell adhesion and morphology.

The inventors have examined the regulatory role of the Sdc1 core protein, in particular the ectodomain, on breast carcinoma cells. The working model depicts Sdc1 as a critical regulator of cell proliferation and invasion of mammary carcinoma cells. Data indicate that the proliferation and morphogenesis of either normal or tumorigenic breast epithelial cells in three-dimensional matrices is exquisitely dependent on Sdc1. Part of this regulation traces to syndecan regulation of signaling downstream integrins, which control polarity vs. invasion of the cells, extravasation of blood-borne metastatic cells from the blood system and their invasion into a target tissue, and also the proliferation and survival of the tumor cells at that site.

The examples below describe the inventors findings that antibodies to the ectodomain of Sdc1 blocked proliferation of cancer cells. In addition, in contrast to the earlier reports of Mali et al. (1994), the inventors have found that the regulatory activity that they describe traces to an epitope present in the protein comprising the extracellular domain of Sdc1 and is retained by heparan-sulfate free Sdc1. Thus, it is proposed to use syndecan 1 ectodomain and agents that bind thereto for the treatment of cancer. The details of the invention are provided below.

1. SYNDECAN 1 PEPTIDES OR POLYPEPTIDES

Sdc1 is highly expressed at the basolateral surface of epithelial cells where it is thought to interact with the actin cytoskeleton and to modulate cell adhesion and growth factor signaling (Bernfield et al., 1999; Rapraeger et al., 1986; Kim et al., 1994; Sanderson and Bemfield, 1988). In experimental studies of malignant transformation, Sdc1 expression is associated with the maintenance of epithelial morphology, anchorage-dependent growth and inhibition of invasiveness. Alterations in syndecan expression during development (Sun et al., 1998) and in transformed epithelia (Inki and Jalkanen, 1996; Bayer-Garner et al., 2001) are associated with an epithelial-mesenchymal transformation with attendant alterations in cell morphology, motility, growth and differentiation. Transfection of epithelial cells with anti-sense mRNA for Sdc l or downregulation of Sdc1 expression by androgen-induced transformation results in an epithelial to mesenchymal transformation and increased invasion (Leppa et al., 1992; Kato et al., 1995; Leppa et al., 1991). The loss of E-cadherin under these circumstances has long suggested a coordinate regulation of Sdc 1 and E-cadherin expression (Sun et al., 1998); Leppa et al., 1996). These studies, as well as others, indicate that there appears to be a threshold requirement for syndecan expression to elicit its biological activity. Sdc1 is downregulated in a number of epithelial cancers and in pre-malignant lesions of the oral mucosa (Soukka et al., 2000) and uterine cervix (Inki et al., 1994; Rintala et al., 1999; Nakanishi et al., 1999), and its loss may be an early genetic event contributing to tumor progression (Sanderson, 2001; Numa et al., 2002; Hirabayashi et al., 1998). Loss of Sdc1 correlates with a reduced survival in squamous cell carcinoma of the head, neck and lung (Anttonen et al., 1999; Inki et al., 1994; Nackaerts et al., 1997), laryngeal cancer (Pulkkinen et al., 1997; Klatka, 2002), malignant mesothelioma (Kumar-Singh et al., 1998) and multiple myeloma (Sanderson, 2002) and a high metastatic potential in hepatocellular and colorectal carcinomas (Matsumoto et al., 1997; Fujiya et al., 2001; Levy et al., 1997; Levy et al., 1996). Downregulation of syndecan-2 and -4 expression has also been observed in certain human carcinomas (Nackaerts et al., 1997; Park et al., 2002; Mundhenke et al., 2002; Crescimanno et al., 1999), but the functional consequences of these alterations in expression are less clear.

In contrast to the general notion that the syndecan may be an inhibitor of carcinogenesis, Sdc1 also demonstrates tumor promoter function. Sdc1 supplements Wnt-1 induced tumorigenesis of the mouse mammary gland (Alexander et al., 2000) and promotes the formation of metastases in mouse lung squamous carcinoma cells (Hirabayashi et al., 1998). Enhanced Sdc l expression has also been observed in pancreatic (Conejo et al., 2000), gastric (Wiksten et al., 2001) and breast (Burbach et al., 2003; Stanley et al., 1999; Barbareschi et al., 2003) carcinomas and this overexpression correlates with increased tumor aggressiveness and poor clinical prognosis. This duality in the role of Sdc1 in tumorigenesis may reflect tissue and/or tumor stage-specific function, or reflect the multiple functions of this PG.

Sanderson was the first to demonstrate a role for Sdc1 in tumor cell migration by examining the invasion of myeloma cells into collagen gels (Liu et al., 1998). Ectopic expression of Sdc1 in syndecan-deficient myeloma cells had the striking effect of curtailing invasion, whereas the expression of other cell surface heparan sulfate PGs (e.g., glypican) was without effect. Using chimeras derived from these two proteins, Sanderson showed that the activity of the syndecan is preserved when its ectodomain alone is expressed as a glycosyl-phosphatidylinositol (GPI)-linked protein at the cell surface. Although clearly responsible for binding the collagen matrix via its attached heparan sulfate chains, the anti-invasive activity of the syndecan requires yet an additional interaction that traces to a site in the extracellular domain of the core protein itself. The mechanism by which the ectodomain site influences the invasion of the myeloma cells is unknown, but its interaction with other cell surface receptors in a “co-receptor” role is one possibility. More recently, ectopic expression of Sdc1 has also been shown to curtail the invasion of hepatocellular carcinoma cells into a collagen matrix (Ohtake et al., 1999).

In a recent study examining Sdc1 in epithelial cells, we have shown a critical role for Sdc1 in αᵥβ₃ integrin signaling activity (Beauvais and Rapraeger, 2003). Most mammary carcinoma cells express multiple syndecan family members (e.g., syndecan-1, syndecan-2, syndecan-4) as well as other cell surface heparan sulfate PGs (e.g., glypican-1). Because adhesion via heparan sulfate to matrix ligands is likely to simultaneously involve all of these receptors, the use of core protein-specific antibodies is the only reliable way to study the adhesion-signaling role of a specific syndecan. This also allows investigation of the syndecan-mediated adhesion without ligand engagement by integrins that would otherwise also participate in binding to a matrix ligand. Employing this procedure with MDA-MB-231 cells, a highly invasive human mammary carcinoma cell line that endogenously expresses syndecans-1 and -4, demonstrates that the cells strongly adhere to mAb B-B4 (specific for human Sdc1) but fail to spread in response to the syndecan ligation. This result was intriguing for several reasons. First, these cells are mammary epithelial cells—a cell type in which it is known Sdc1 functions to regulate cell morphology (Leppa et al., 1992). Second, it contrasts with studies with other cell types (e.g., Raji, myeloma, COS, etc.) in which adherence via Sdc1 leads to an active signaling and spreading process. Finally, the cells do spread when plated on the HepII domain of fibronectin, which can engage multiple cell surface PGs, or in response to syndecan-4 ligation. As MDA-MB-231 cells adherent via syndecan-4 spread and cells adherent via Sdc1 do not, these results indicate that syndecan-1 and -4 may trigger different signaling pathways and that the Sdc1 adhesion-signaling pathway may be missing or selectively shut-off.

Despite their initial failure to spread when adherent via Sdc1 alone, MDA-MB-231 cells do spread if treated with 1 mM manganese (Mn²⁺). As Mn²⁺ induces conformational shifts that mimic the physiological activation of β₁ and β₃ integrins, these results indicated that it might be necessary to activate one of the β1 integrins, or the αᵥβ₃ integrin, on these cells as an integrin partner working in collaboration with Sdc1 ligation.

The identity of which integrin(s) cooperate with Sdc1 was determined using modulatory antibodies to induce conformational shifts that mimic the active or inactive states of a particular integrin. These studies revealed that activation of β₁ integrins is not required for Sdc1 mediated cell spreading. In fact, inhibition of β₁ integrins induces cell spreading—presumably by releasing the integrins'-dependent suppression of the syndecan. Use of other integrin modulatory antibodies demonstrated that Sdc1 collaborates with the αᵥβ₃ integrin to initiate a positive spreading signal and that trans-dominant inhibition arising from an α₂β₁-αᵥβ₃ integrin cross-talk prevents this signal from being generated. Intriguingly, cooperative signaling via αᵥβ₃ integrins occurs in the absence of an integrin ligand (i.e., Sdc1 ligation is sufficient)—a contention supported by evidence from other published works that unligated integrins are capable of transmitting intracellular signals both in vitro and in vivo (Brooks et al., 1994); Domanico et al., 1997; Brassard et al., 1999; Kuzuya et al., 1999; Stupack et al., 2001; Bachelder et al., 1999; Lewis et al., 2002; Iba et al., 2000; Thodeti et al., 2003). What role, Sdc1 plays, if any, in the allosteric activation of αᵥβ₃ integrins is presently unknown. However, current work indicates that ectopic expression of murine Sdc1 can block β₁ integrin function in human mammary carcinoma cells. As such, the failure of MDA-MB-231 cells to initially spread may trace to sub-optimal levels of cell surface Sdc1 expression and as a consequence, hyperactivation of β₁ integrins. In fact, MDA-MB-231 cells overexpressing human or murine Sdc1 are capable of spreading in response to Sdc1 ligation in the absence of β₁ integrin blockade (Beauvais et al., 2004).

An important feature of the syndecan necessary for signaling appears to be its ectodomain. Specific evidence supporting a role for the ectodomains of Sdc1 and -4 in cell adhesion has previously been described in our laboratory (McFall and Rapraeger, 1997; McFall and Rapraeger, 1998). Soluble murine Sdc1 ectodomain competitively inhibits MDA-MB-231 cell spreading and in short-term migration assays in a dose-dependent manner (Beauvais and Rapraeger, 2003; Beauvais et al., 2004). This competition is specific for Sdc1, as soluble syndecan-4 ectodomain fails to compete. These results suggest that soluble murine Sdc1 competes with the endogenous human syndecan for a critical cell surface interaction required for signaling during cell spreading. Indeed, expression of a Sdc1 mutant, in which a portion of the ectodomain is deleted, fails to signal spreading, while deletion of the Sdc1 transmembrane and cytoplasmic tail is without effect (Beauvais et al., 2004). These results indicate a regulatory role for the Sdc1 ectodomain in the regulation of epithelial cell morphology, in agreement with earlier published works Kato et al., 1995; Mali et al., 1994).

A related integrin-regulatory function of Sdc1 is observed with the aᵥβ₅ integrin (McQuade et al., 2006). B82L mouse fibroblasts expressing this integrin rely on the co-expression of Sdc1 for integrin activation. If Sdc1 expression is silenced, the integrin is not active and the cells fail to bind and/or spread on ligands for this integrin such as vitronectin. Similar to the regulation of the βᵥβ₃ integrin by Sdc1, αᵥβ₅ integrin activity can be rescued by expression of a Sdc1 mutant containing only the ectodomain of Sdc1 anchored to the membrane by a lipid-tail. Sdc1 mutants in which the ectodomain has been replaced by sequences of other proteins do not rescue. The integrin can also be inactivated by adding competiting concentrations of recombinant Sdc1 ectodomain, which ostensibly competes with the assembly of the Sdc1 and the αᵥβ₅ integrin into a complex at the cell surface. Indeed, immunoprecipitation of Sdc1 from the B82L cells co-precipitates the integrin with the Sdc1 and this co-precipitation is disrupted by competing concentration of Sdc1 ectodomain (McQuade et al., 2006). Thus, the active site in the Sdc1 responsible for activating this integrin also appears to reside in the extracellular domain of the protein.

A. Structural Features

The sequence of human syndecan 1 is provided in SEQ ID NO:1 (Accession No. NM_{—}002997). Syndecan 1 ectodomain is comprised of residues 18 to 251 of SEQ ID NO:1 (SEQ ID NO:3). Full length ectodomain, as well as fragments of the ectodomain ranging in size from 5 to about 100, 15 to 50, and 25 to 40 residues, all of which retain the anti-cancer function exhibited by the entire ectodomain also are contemplated. The peptides may be generated synthetically or by recombinant techniques, and are purified according to known methods, such as precipitation (e.g., ammonium sulfate), HPLC, ion exchange chromatography, affinity chromatography (including immunoaffinity chromatography) or various size separations (sedimentation, gel electrophoresis, gel filtration). In general, they will lack heparan sulfate residues.

The peptides may be labeled using various molecules, such as fluorescent, chromogenic or colorimetric agents. The peptides may also be linked to other molecules, including other anti-cancer agents. The links may be direct or through distinct linker molecules. The linker molecules in turn may be subject, in vivo, to cleavage, thereby releasing the agent from the peptide. Peptides may also be rendered multimeric by linking to larger, and possibly inert, carrier molecules.

B. Variants or Analogs of Syndecan 1

i) Substitutional Variants

It also is contemplated in the present invention that variants or analogs of syndecan 1 ectodomain may exhibit anti-cancer activity. Peptide and polypeptide sequence variants of syndecan 1 ectodomain, primarily making conservative amino acid substitutions to SEQ ID NO:1, may provide improved compositions. Substitutional variants typically contain the exchange of one amino acid for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

The following is a discussion based upon changing of the amino acids of a peptide to create an equivalent, or even an improved, second-generation molecule. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a peptide that defines that peptide's biological functional activity, certain amino acid substitutions can be made in a protein sequence, and its underlying DNA coding sequence, and nevertheless obtain a peptide with like properties. It is thus contemplated by the inventors that various changes may be made in the DNA sequences coding the peptide without appreciable loss of their biological utility or activity, as discussed below.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant peptide, which in turn defines the interaction of the peptide with other molecules.

Each amino acid has been assigned a hydropathic index on the basis of its hydrophobicity and charge characteristics (Kyte and Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a peptide with similar biological activity, i.e., still obtain a biological functionally equivalent protein. In making such changes, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2) glutamine (+0.2); glycine (0); threonine (−0.4); proline (−0.5±1); alanine (−0.5); histidine *−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still obtain a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

Another embodiment for the preparation of polypeptides according to the invention is the use of peptide mimetics. Mimetics are peptide containing molecules that mimic elements of protein secondary structure (Johnson et al., 1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used, in conjunction with the principles outline above, to engineer second generation molecules having many of the natural properties of syndecans, but with altered and even improved characteristics.

ii) Altered Amino Acids

The present invention may employ peptides that comprise modified, non-natural and/or unusual amino acids. A table of exemplary, but not limiting, modified, non-natural and/or unusual amino acids is provided herein below. Chemical synthesis may be employed to incorporated such amino acids into the peptides of interest.

TABLE 1
Modified, Non-Natural and Unusual Amino Acids

| Abbr. | Amino Acid | Abbr. | Amino Acid |
|---|---|---|---|
| Aad | 2-Aminoadipic acid | EtAsn | N-Ethylasparagine |
| BAad | 3-Aminoadipic acid | Hyl | Hydroxylysine |
| BAla | beta-alanine, beta-Amino- | AHyl | allo-Hydroxylysine |
| | propionic acid | 3Hyp | 3-Hydroxyproline |
| Abu | 2-Aminobutyric acid | 4Hyp | 4-Hydroxyproline |
| 4Abu | 4-Aminobutyric acid, | Ide | Isodesmosine |
| | piperidinic acid | Aile | allo-Isoleucine |
| Acp | 6-Aminocaproic acid | MeGly | N-Methylglycine, sarcosine |
| Ahe | 2-Aminoheptanoic acid | MeIle | N-Methylisoleucine |
| Aib | 2-Aminoisobutyric acid | MeLys | 6-N-Methylysine |
| BAib | 3-Aminoisobutyric acid | MeVal | N-Methylvaline |
| Apm | 2-Aminopimelic acid | Nva | Norvaline |
| Dbu | 2,4-Diaminobutyric acid | Nle | Norleucine |
| Des | Desmosine | Orn | Ornithine |
| Dpm | 2,2′-Diaminopimelic acid | | |
| Dpr | 2,3-Diaminopropionic acid | | |
| EtGly | N-Ethylglycine | | |

iii) Mimetics

In addition to the variants discussed above, the present inventors also contemplate that structurally similar compounds may be formulated to mimic the key portions of syndecan 1 ectodomain. Such compounds, which may be termed peptidomimetics, may be used in the same manner as the peptides of the invention and, hence, also are functional equivalents.

Certain mimetics that mimic elements of protein secondary and tertiary structure are described in Johnson et al. (1993). The underlying rationale behind the use of peptide mimetics is that the peptide backbone of proteins exists chiefly to orient amino acid side chains in such a way as to facilitate molecular interactions, such as those of antibody and/or antigen. A peptide mimetic is thus designed to permit molecular interactions similar to the natural molecule.

Some successful applications of the peptide mimetic concept have focused on mimetics of β-turns within proteins, which are known to be highly antigenic. Likely β-turn structure within a polypeptide can be predicted by computer-based algorithms, as discussed herein. Once the component amino acids of the turn are determined, mimetics can be constructed to achieve a similar spatial orientation of the essential elements of the amino acid side chains.

Other approaches have focused on the use of small, multidisulfide-containing proteins as attractive structural templates for producing biologically active conformations that mimic the binding sites of large proteins (Vita et al., 1998). A structural motif that appears to be evolutionarily conserved in certain toxins is small (30-40 amino acids), stable, and high permissive for mutation. This motif is composed of a beta sheet and an alpha helix bridged in the interior core by three disulfides.

Beta II turns have been mimicked successfully using cyclic L-pentapeptides and those with D-amino acids (Weisshoff et al., 1999). Also, Johannesson et al. (1999) report on bicyclic tripeptides with reverse turn inducing properties.

Methods for generating specific structures have been disclosed in the art. For example, alpha-helix mimetics are disclosed in U.S. Pat. Nos. 5,446,128; 5,710,245; 5,840,833; and 5,859,184. Theses structures render the peptide or protein more thermally stable, also increase resistance to proteolytic degradation. Six, seven, eleven, twelve, thirteen and fourteen membered ring structures are disclosed.

Methods for generating conformationally restricted beta turns and beta bulges are described, for example, in U.S. Pat. Nos. 5,440,013; 5,618,914; and 5,670,155. Beta-turns permit changed side substituents without having changes in corresponding backbone conformation, and have appropriate termini for incorporation into peptides by standard synthesis procedures. Other types of mimetic turns include reverse and gamma turns. Reverse turn mimetics are disclosed in U.S. Pat. Nos. 5,475,085 and 5,929,237, and gamma turn mimetics are described in U.S. Patents 5,672,681 and 5,674,976.

C. Fusion Proteins

Another variant is a fusion protein. This molecule generally has all or a substantial portion of the original molecule, in this case a peptide or polypeptide from the ectodomain of syndecan 1, linked at the N- or C-terminus to all or a portion of a second peptide or polypeptide. For example, fusions may employ leader sequences from other species to permit the recombinant expression of a protein in a heterologous host. Another useful fusion includes the addition of a immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes, glycosylation domains, cellular targeting signals or transmembrane regions. Of particular interest are peptide permeant motifs that improve peptides transfer through membranes. Such mofits include those from TAT and R9:
TAT=RKKRRQRRR (Schwarze et al., 2000; Becker-Hapak et al., 2001; Denicourt and Dowdy, 2003)
R9=RRRRRRRR (Wender et al., 2000)

There also may be instances where a greater degree of intracellular specificity is desired. For example, with targeting nuclear proteins, RNA, DNA or cellular proteins or nucleic acids that are subsequently processed. Thus, one preferably uses localization sequences for such targets. Localization sequences have been divided into routing signals, sorting signals, retention or salvage signals and membrane topology-stop transfer signals (Yellon et al., 1992). For example, there are signals to target the endoplasmic reticulum (Munro, et al., 1987), the nucleus (Lanford et al., 1986; Stanton et al., 1986; Harlow et al., 1985), the nucleolar region (Kubota et al., 1989; and Siomi et al., 1988), the endosomal compartment (Bakke et al., 1990), mitochondria (Yellon et al., 1992) and liposomes (Letourneur et al., 1992). One nuclear targeting sequence may be the SV40 nuclear localization signal.

D. Purification of Proteins

It may be desirable to purify syndecan 1 peptides or polypeptides, variants, peptide-mimics or analogs thereof. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

E. Peptide Synthesis

Syndecan 1-related peptides may be generated synthetically for use in various embodiments of the present invention. Because of their relatively small size, the peptides of the invention can be synthesized in solution or on a solid support in accordance with conventional techniques. Various automatic synthesizers are commercially available and can be used in accordance with known protocols. See, for example, Stewart & Young, (1984); Tam et al., (1983); Merrifield, (1986); Barany and Merrifield (1979), each incorporated herein by reference. Short peptide sequences, or libraries of overlapping peptides, usually from about 6 up to about 35 to 50 amino acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

### 2. SYNDECAN 1-ENCODING NUCLEIC ACIDS

Important aspects of the present invention concern isolated DNA segments and recombinant vectors encoding syndecan 1 and peptides thereof, the creation and use of recombinant host cells through the application of DNA technology, that express syndecan 1 or peptides thereof, and biologically functional equivalents thereof. The human syndecan 1 DNA sequence is shown in SEQ ID NO:2.

The present invention concerns DNA segments, isolatable from mammalian cells, such as mouse, rat or human cells, that are free from total genomic DNA and that encode a syndecan 1 ectodomain or peptides thereof. As used herein, the term “DNA segment” refers to a DNA molecule that has been isolated free of total genomic DNA of a particular species. Therefore, a DNA segment encoding syndecan 1 refers to a DNA segment that contains wild-type, polymorphic or mutant syndecan 1 coding sequences yet is isolated away from, or purified free from, total mammalian genomic DNA. Included within the term “DNA segment” are DNA segments and also recombinant vectors, including, for example, plasmids, cosmids, phage, viruses, and the like.

In particular embodiments, the invention concerns isolated DNA segments and recombinant vectors incorporating DNA sequences that encode a syndecan 1 ectodomain (SEQ ID NO:4), a peptide, or a biologically functional equivalent of syndecan 1. The term “biologically functional equivalent” is well understood in the art and is further defined in detail herein. Accordingly, sequences that have about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, and any range derivable therein, such as, for example, about 70% to about 80%, and more preferably about 81% and about 90%; or even more preferably, between about 91% and about 99%; of amino acids that are identical or functionally equivalent to the amino acids of SEQ ID NOS:1 or 3 or any analog or variant thereof provided the biological activity of the protein is maintained. In particular embodiments, the biological activity of a syndecan 1 ectodomain.

It will also be understood that amino acid and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological protein, polypeptide or peptide activity where an amino acid sequence expression is concerned. The addition of terminal sequences particularly applies to nucleic acid sequences that may, for example, include various non-coding sequences flanking either of the 5′ or 3′ portions of the coding region or may include various internal sequences, i.e., introns, which are known to occur within genes.

### 3. SCREENING ASSAYS

The present invention also contemplates the screening of compounds, e.g., peptides, peptide-mimics, variants, analogs or small molecules, for ability to interact with syndecan 1 ectodomain. In the screening assays of the present invention, the candidate substance may first be screened for basic biochemical activity, e.g., binding to a syndecan 1 ectodomain, and then tested for its ability to inhibit cancer.

Thus, present invention provides methods of screening for agents that bind syndecan 1. In an embodiment, the present invention is directed to a method of:
(a) providing a syndecan 1 ectodomain;
(b) contacting said syndecan 1 ectodomain with a candidate substance; and
(c) determining the binding of the candidate substance to the syndecan 1 ectodomain,
   wherein binding to syndecan 1 identifies the compound as a putative anti-cancer agent.

Measuring binding to syndecan 1 ectodomain may be direct, by identifying a syndecan 1-candidate complex, or by identifying labeled candidate associated with syndecan 1 ectodomain. In still yet other embodiments, one would look at the effect of a candidate on pain in an appropriate model.

A. Modulators

As used herein, the term “candidate substance” refers to any molecule that may potentially modulate bind to syndecan 1 ectodomain. The candidate substance may be a peptide, or a small molecule inhibitor, or even a nucleic acid molecule. It may prove to be the case that the most useful pharmacological compounds will be compounds that are structurally related to compounds which interact naturally with syndecan 1. Creating and examining the action of such molecules is known as “rational drug design,” and include making predictions relating to the structure of target molecules.

The goal of rational drug design is to produce structural analogs of biologically active polypeptides or target compounds. By creating such analogs, it is possible to fashion drugs which are more active or stable than the natural molecules, which have different susceptibility to alteration or which may affect the function of various other molecules. In one approach, one would generate a three-dimensional structure for a molecule like syndecan 1, and then design a molecule for its ability to interact with syndecan 1, or the ectodomain thereof. This could be accomplished by x-ray crystallography, computer modeling or by a combination of both approaches.

It also is possible to use antibodies to ascertain the structure of a target compound or inhibitor. In principle, this approach yields a pharmacore upon which subsequent drug design can be based. An example of such an approach is to use syndecan 1 ectodomain as a model, and then create a molecule that would mimic the anti-syndecan 1 antibody. On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Antibodies to syndecan 1 ectodomain are also useful in and of themselves. The term “antibody” is used to refer to any antibody-like molecule that has an antigen binding region, and/or includes antibody fragments such as Fab′, Fab, F(ab′)₂, single domain antibodies (DABs), Fv, scFv (single chain Fv), and/or the like. The techniques for preparing and/or using various antibody-based constructs and/or fragments are well known in the art. Means for preparing and/or characterizing antibodies are also well known in the art (see, e.g., Antibodies: A Laboratory Manual, Cold Spring Harbor Laboratory, 1988; incorporated herein by reference).

As used herein, the term “antibody” is intended to refer broadly to any immunologic binding agent such as IgG, IgM, IgA, IgD and/or IgE, as well as polyclonal or monoclonal antibodies. Monoclonal antibodies (MAbs) are recognized to have certain advantages, e.g., reproducibility and/or large-scale production, and/or their use is generally preferred. “Humanized” antibodies are also contemplated, where non-human constant region sequences are replaced with human sequences while leaving the binding specificity relatively unchanged. These chimeric antibodies may be from mouse, rat, and/or other non-human species, and engineered to contain human constant domains. U.S. Pat. No. 5,482,856.

Candidate compounds also may include fragments or parts of naturally-occurring compounds or may be found as active combinations of known compounds which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be a polypeptide, polynucleotide, small molecule inhibitor or any other compounds that may be designed through rational drug design starting from known inhibitors of hypertrophic response.

It will, of course, be understood that all the screening methods of the present invention are useful in themselves notwithstanding the fact that effective candidates may not be found. The invention provides methods for screening for such candidates, not solely methods of finding them.

B. In Vitro Assays

A quick, inexpensive and easy assay to run is a syndecan 1 ectodomain binding assay. Binding of a molecule to syndecan 1 ectodomain may, in and of itself, be inhibitory, due to steric, allosteric or charge-charge interactions. This assay can be performed in solution or on a solid phase and can be utilized as a first round screen to rapidly eliminate certain compounds before moving into more sophisticated screening assays.

The target (e.g., syndecan 1 ectodomain) may be either free in solution, fixed to a support, expressed in or on the surface of a cell. Either the target or the compound may be labeled, thereby permitting determination of binding. Competitive binding assays can be performed in which syndecan 1 ectodomain is used. The syndecan 1 ectodomain may be labeled, or the candidate may be labeled. One may measure the amount of free label versus bound label to determine binding or inhibition of binding.

A technique for high throughput screening of compounds is described in WO 84/03564. Large numbers of small peptide test compounds are synthesized on a solid substrate, such as plastic pins or some other surface. The peptide test compounds are reacted with, for example, syndecan 1 ectodomain and washed. Bound polypeptide is detected by various methods.

C. In Cyto Assays

Various cells that express syndecan 1 endogenously or are transfected with cDNA for all or part of the syndecan 1 gene, can be utilized for screening of candidate substances. Exemplary cells include, but are not limited to yeast cells, bacterial cells, COS cells, HEK293 cells. Depending on the assay, culture may be required. Labeled candidate substances or competitive inhibitors may be contacted with the cell and binding assessed. Various readouts for binding of candidate substances to cells may be utilized, including fluorescent microscopy and FACS.

D. In Vivo Assays

The present invention particularly contemplates the use of various animal models. For example, various animal models of cancer may be used to determine if candidate substances that bind to syndecan 1 ectodomain affect the ability of a cancer cell to growth, proliferate, divide, invade tissue or metastasize.

Treatment of these animals with test compounds will involve the administration of the compound, in an appropriate form, to the animal. Administration will be by any route that could be utilized for clinical or non-clinical purposes, including but not limited to oral, nasal, buccal, or even topical. Alternatively, administration may be by oral, sublingual, intratracheal instillation, bronchial instillation, intradermal, subcutaneous, intramuscular, intraperitoneal, intratumoral or intravenous injection. Specifically contemplated are oral administration and systemic intravenous injection.

### 4. ENGINEERING EXPRESSION CONSTRUCTS

In certain embodiments, the present invention involves the production of syndecan 1 or its ectodomain. Such methods rely upon expression constructs containing a syndecan 1 or ectodomain coding region and the means for its expression, plus elements that permit replication of the constructs. A variety of elements and vector types are discussed below.

A. Selectable Markers

In certain embodiments of the invention, expression constructs of the present invention contain nucleic acid constructs whose expression may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants. For example, genes that confer resistance to neomycin, puromycin, hygromycin, DHFR, GPT, zeocin and histidinol are useful selectable markers. Alternatively, enzymes such as herpes simplex virus thymidine kinase (tk) may be employed. Immunologic markers also can be employed. The selectable marker employed is not believed to be important, so long as it is capable of being expressed simultaneously with the nucleic acid encoding a gene product. Further examples of selectable markers are well known to one of skill in the art and include reporters such as EGFP, β-gal or chloramphenicol acetyltransferase (CAT).

B. Polyadenylation Signals

One will typically desire to include a polyadenylation signal to effect proper polyadenylation of the transcript. The nature of the polyadenylation signal is not believed to be crucial to the successful practice of the invention, and any such sequence may be employed such as human or bovine growth hormone and SV40 polyadenylation signals. Also contemplated as an element of the expression cassette is a terminator. These elements can serve to enhance message levels and to minimize read through from the cassette into other sequences.

C. Control Regions

Throughout this application, the term “expression construct” is meant to include any type of genetic construct containing a nucleic acid coding for the peptide of interest. The nucleic acid encoding the peptide is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation.

For the purpose of recombinant production, prokaryotic (bacteria) and lower eukaryotic organisms (yeast) can be used. Commercial vectors and expression systems, including appropriate host cells and methods for transformation and culture, are well known to those of skill in the art.

Promoters generally refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription.

The particular promoter employed to control the expression of a nucleic acid sequence of interest is not believed to be important, so long as it is capable of directing the expression of the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter.

In various embodiments, the human cytomegalovirus (CMV) immediate early gene promoter, the SV40 early promoter, the Rous sarcoma virus long terminal repeat, β-actin, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized. Other viral promoters that may be used depending on the desired effect include adenovirus promoters such as from the E1A, E2A, or MLP region, AAV LTR, HSV-TK, and avian sarcoma virus. Promoters may also be tissue specific or inducible.

Enhancers are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

D. Vectors

Various vector systems can be used in accordance with the present invention to prepare syndecan 1 peptides and polypeptides. One class of vector systems is the viral vectors. Adenovirus is particularly suitable for use as a gene transfer vector because of its mid-sized DNA genome, ease of manipulation, high titer, wide target-cell range, and high infectivity. The roughly 36 kB viral genome is bounded by 100-200 base pair (bp) inverted terminal repeats (ITR), in which are contained cis-acting elements necessary for viral DNA replication and packaging, much of which can be removed, making room for exogenous genes. It also can package approximately 110% of the normal genome.

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes—gag, pol and env—that code for capsid proteins, polymerase enzyme, and envelope components, respectively. A sequence found upstream from the gag gene, termed ψ, functions as a signal for packaging of the genome into virions. Two long terminal repeat (LTR) sequences are present at the 5′ and 3′ ends of the viral genome. These contain strong promoter and enhancer sequences and also are required for integration in the host cell genome (Coffin, 1990).

AAV utilizes a linear, single-stranded DNA of about 4700 base pairs. Inverted terminal repeats flank the genome. Two genes are present within the genome, giving rise to a number of distinct gene products. The first, the cap gene, produces three different virion proteins (VP), designated VP-1, VP-2 and VP-3. The second, the rep gene, encodes four non-structural proteins (NS). One or more of these rep gene products is responsible for transactivating AAV transcription. AAV is not associated with any pathologic state in humans. Interestingly, for efficient replication, AAV requires “helping” functions from viruses such as herpes simplex virus I and II, cytomegalovirus, pseudorabies virus and, of course, adenovirus. The best characterized of the helpers is adenovirus, and many “early” functions for this virus have been shown to assist with AAV replication. Low level expression of AAV rep proteins is believed to hold AAV structural expression in check, and helper virus infection is thought to remove this block.

Other viral vectors may be employed as expression constructs in the present invention. Vectors derived from viruses such as vaccinia virus (Ridgeway, 1988; Baichwal and Sugden, 1986; Coupar et al., 1988) canary pox virus, and herpes viruses may be employed. These viruses offer several features for use in gene transfer into various mammalian cells.

Several non-viral methods for the transfer of expression constructs into cells are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979), cell sonication (Fechheimer et al., 1987), gene bombardment using high velocity microprojectiles (Yang et al., 1990), and receptor-mediated transfection (Wu and Wu, 1987; Wu and Wu, 1988).

In a particular embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). The addition of DNA to cationic liposomes causes a topological transition from liposomes to optically birefringent liquid-crystalline condensed globules (Radler et al., 1997). These DNA-lipid complexes are potential non-viral vectors for use in gene therapy.

In another embodiment of the invention, the expression construct may simply consist of naked recombinant DNA or plasmids. Transfer of the construct may be performed by any of the methods mentioned above which physically or chemically permeabilize the cell membrane. This is applicable particularly for transfer in vitro, however, it may be applied for in vivo use as well. Dubensky et al., (1984) successfully injected polyomavirus DNA in the form of CaPO₄ precipitates into liver and spleen of adult and newborn mice demonstrating active viral replication and acute infection. Benvenisty and Neshif (1986) also demonstrated that direct intraperitoneal injection of CaPO₄ precipitated plasmids results in expression of the transfected genes. It is envisioned that DNA encoding a CAM also may be transferred in a similar manner in vivo and express CAM.

### 5. PHARMACEUTICAL FORMULATIONS

Pharmaceutical formulations of the present invention comprise an effective amount of a syndecan 1 ectodomain dissolved or dispersed in a pharmaceutically acceptable carrier. The phrases “pharmaceutical or pharmacologically acceptable” refer to compositions that do not produce an adverse, allergic or other untoward reaction when administered to an animal, such as, for example, a human, as appropriate. The preparation of such pharmaceutical compositions are known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art. Except insofar as any conventional carrier is incompatible with the active ingredient, its use in the therapeutic or pharmaceutical compositions is contemplated.

The pharmaceuticals of the present invention may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g., aerosol), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The pharmaceuticals may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In certain embodiments, the compositions are prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain preferred embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

### 6. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

### Example 1

### Materials and Methods

Materials. Matrigel (10 mg/ml, BD biosciences, #354234) was used for three dimensional (3D) culture. Polyclonal antibodies against Sdc1, syndecan-4 and GST were raised in rabbit and dissolved in PBS. Rabbit IgG (whole molecule) was purchased from Jachson Immuno Research (#011-000-003). Mouse anti-human Sdc1 ectodomain monoclonal antibody B-B4 was purchased from Serotech (MCA681).

Cell Culture. The MDA-MB-231 mammary carcinoma cells were grown in DMEM medium (GIBCO BRL, St. Louis, Mo.) containing 10% FBS. The MCF-7 cells were grown in DMEM medium containing 10% FBS and 10 μg/ml insulin. The MCF-10A cells were grown in DMEM:F12 50:50 mix (15 mM Hepes and L-glut) medium (GIBCO BRL) containing 5% horse serum, 10 μg/ml insulin, 0.5 μg/ml Hydrocortisone, 0.02 μg/ml EGF.

Full length mouse Sdc1 (FLmS1),GPI linked mS1ED (GPImS1ED) or a construct of mouse Sdc1 in which the heparan sulfate attachment sites are mutated (TDM) were stably transfected into MDA-MB-231 cells and selected with 1.5 mg/ml G418 (Gibco BRL). These stable transfectants were maintained in culture in the presence of G418 and were checked periodically for constant expression of FLmS 1, GPImS1ED or TDM. Cells expressing these transgenes at similar levels were sorted by FACS before the experiments. Cells transfected with the empty pcDNA3 vector (NEO) were used as control.

3D Culture. For 3D culture, cells were suspended with trypsin and embedded into Matrigel™ as single cells (4×10⁵/ml). Monoclonal antibody B-B4 specific for Sdc1, nonspecific rabbit IgG or rabbit polyclonal antibodies against Sdc1, Sdc4, or GST were suspended in PBS and added into the matrigel at the same time. After the matrigel was polymerized at 37° C. for 30-60 min, culture media with or without the same treatments were added into each well. A range of 50-200 μg/ml antibodies were used. The same volume of PBS without antibody was used as an additional control. For culture in 2D culture, cells were released with trypsin and seeded on tissue culture plastic dishes in the presence of serum. Cultures were grown for up to 6 days.

Silencing of human Sdc1 with specific short interfering RNA (siRNA). Purified, duplexed siRNAs specific for human Sdc1 were purchased from Ambion (Austin, Tex.). The siRNA sequences targeting human Sdc1 were: hsdc1-1, AAGGAGGAATTCTATGCCTGA; hsdc1-2, AAGGAGGAATTCTATGCCTGA; hsdc1-3, AAGGTAAGTTAAGTAAGTTGA. Two hundred and fifty pmols of siRNA mixture was transfected into MDA-MB-231 cells cultured on 35mm tissue culture dishes using Lipofectamine 2000. siRNA was also transfected into MDA-MB-231 cells that had previously been stably transfected with control or FLmS1 or GPImS1ED or TDM plasmids. Transfected cells were cultured for an additional 24 hrs before use.

Assessment of Cell Growth. To quantify cell proliferation, cells grown in matrigel (3D culture) or on top of matrigel (2D culture) were released from the matrigel by 45-60 min incubation at 37° C. with Dispase (BD Biosciences), washed with Dulbecco's modified Eagle's medium (DMEM) 2-3 times and resuspended into 400 ul fresh DMEM for cell counting by hemocytometer. Triplicate wells, replicated in at least three experiments, were used for the quantification.

To determine whether cells were arrested in a particular phase of the cell cycle, cells were first released from the matrigel by adding 400 μl dispase (BD Biosciences) and incubating at 37° C. for 1 hour. On obtaining a cell pellet and washing with PBS, the samples were incubated in 10% CS containing HEPES buffered DMEM (HbDME+10% CS) at 37° C. for 1 hour to allow regeneration of cell surface Sdcl. About 3-5×10⁵ cells were resuspended in 100 μl of ice-cold HbDME+10%CS with or without 1 μg of mouse anti-human Sdc1 mAb B-B4 and incubated on ice for 60 min. Samples were then washed in 1 ml of ice-cold HbDME+10% CS medium and pelleted again. Cells were then resuspended in 100 μl of ice-cold HbDME+10% CS with 0.5 μg of Alexa488-conjugated goat anti-mouse IgG and incubated on ice for 15 min. After washing, cells were resuspended in 1 ml HbDME+10%CS containing 20 μg Hoechst 33342 (Molecular Probes) and incubated at 37° C. for 20 min. Cells were then analyzed on a FACSCalibur flow cytometer (Becton Dickinson) and the data were assessed using Modfit software (Verity Software House).

### Example 2

### Results

The role of Sdc1 in carcinoma cell proliferation was tested using three different human mammary epithelial cell lines: MCF10A mammary epithelial cells, which display normal, non-invasive cell characteristics when cultured in vitro in a three-dimensional (3D) matrigel or type I collagen gel and are not tumorigenic in vivo; MCF-7 mammary carcinoma cells, which are moderately invasive in 3D gels in vitro and mildly tumorigenic in vivo; and MDA-MB-231 mammary carcinoma cells that are highly invasive in 3D gels and are highly tumorigenic in vivo. Analysis of the growth and differentiation of these cells in either collagen or Matrigel™ is similar and the results when examining the role of Sdc1 in their proliferation are also similar in either matrix.

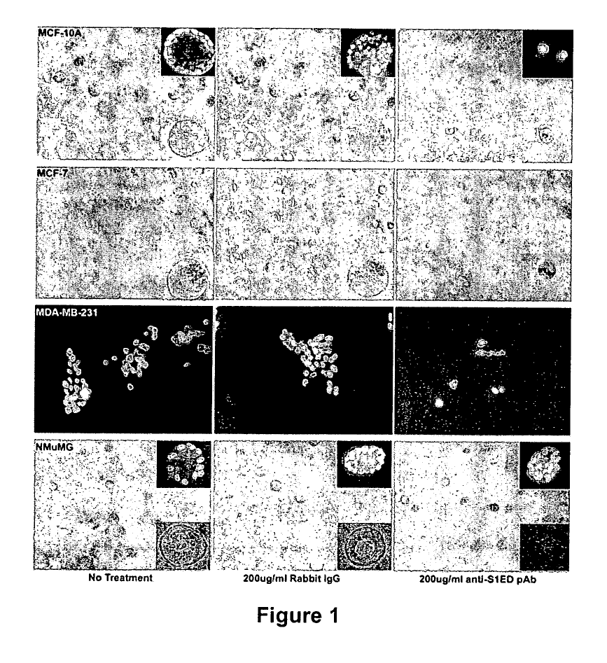

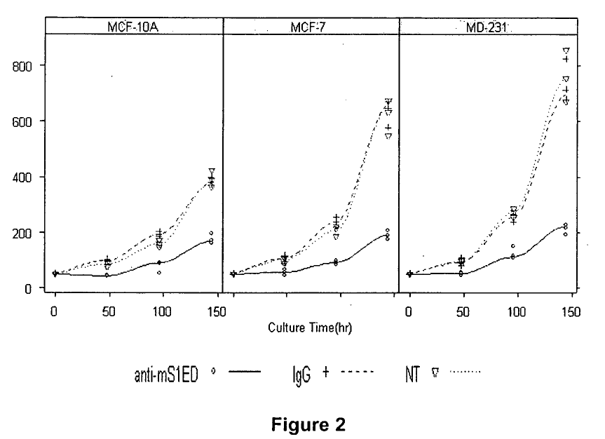

After 6 days, the MCF10A cells began to form quiescent acini with hollow lumens, the MCF-7 cells form cell-filled acini and the MDA-MB-231 cells did not form acini. Rather, they grew as disorganized cords or sheets of cells in matrigel, as has been observed previously. To test for a regulatory role of the Sdc1 extracellular protein domain, each of the three cell types was treated over the 6 day culture period with 50-200 μg/ml of a polyclonal antibody raised in rabbits against recombinant Sdc1 extracellular domain (S1ED). This recombinant immunogen was expressed and purified from bacteria and thus does not bear heparan sulfate or other sugar modifications that are unique to eukaryotic cells. Sdc1-specific antibody blocked the growth of each of the mammary epithelial or carcinoma cell lines, as examination of the cells by microscopy shows that they remain single or as doublets (FIG. 1) and quantification of overall cell numbers after six days showed a dramatic reduction in the cells cultured in the presence of the anti-syndecan antibody (FIG. 2). In contrast, the cells were unaffected by identical concentrations of a nonspecific antibody (anti-GST) or a polyclonal antibody directed against the extracellular domain of syndecan-4. The inventors also found that a monoclonal antibody against the Sdc1 extracellular domain (mAb B-B4) did not have this effect (data not shown), suggesting that the effect is not a general effect of antibody binding the syndecan; rather, the interpretation is that one of the antibodies in the polyclonal preparation is binding to a specific site on the Sdc1 protein that is necessary to regulate cell proliferation. The inventors also found that a normal mouse mammary epithelial cell line (NMuMG cells) is not affected by the Sdc1 polyclonal antibody treatment, despite the fact that these cells do express Sdc1. Although there is no current explanation for this difference, it does indicate that the antibody preparation itself is not having a deleterious effect on the cells.

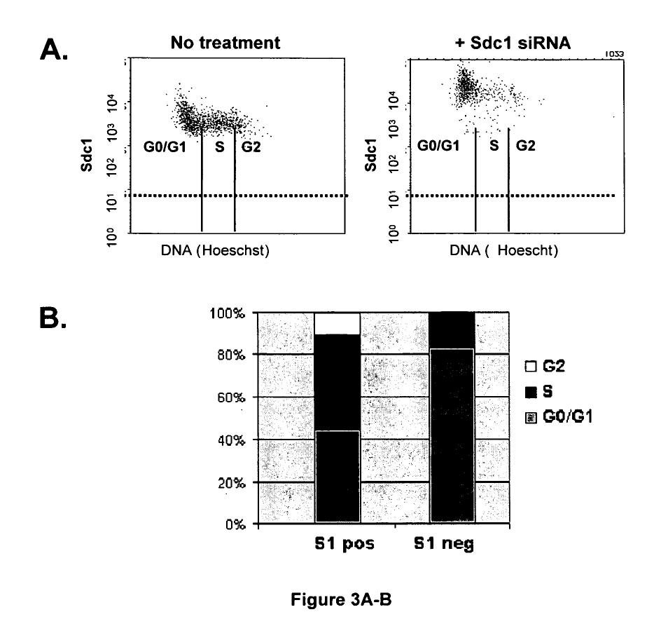

To further confirm this finding, the inventors tested cells in which the expression of Sdc1 is silenced with short interfering RNA (siRNA). This technique uses siRNA oligonucleotides that are specific for human Sdc1, but do not silence mouse Sdc1. Thus, the inventors can test the effect of silencing the endogenous Sdc1 in the cells, and then question which domain of the syndecan protein is critical for the activity by rescuing the cells with mouse Sdc1 that is lacking specific protein regions. MDA-MB-231 carcinoma cells showed reduction in cell proliferation in 3D matrigel when Sdc1 expression is silenced by 80-90% using siRNA (FIGS. 3A-B). Releasing the cells from matrigel and examining them by flow cytometry shows that those cells that have reduced amounts of Sdc1 appear to be arrested in the G₀/G₁ stage of the cell cycle (FIGS. 3A-B)—a common feature of cells that fail to receive or properly process signals leading the activation of the cell cycle and cell division.

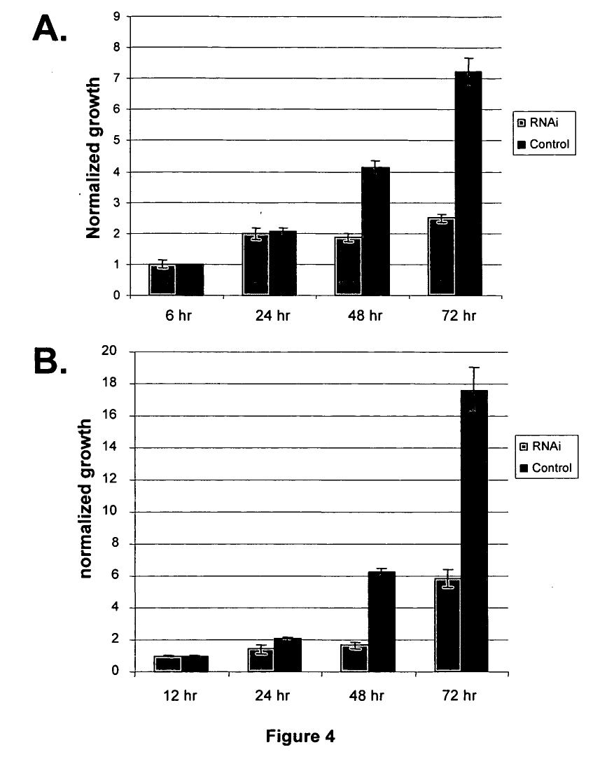

Quantification assays show that silencing Sdc1 expression using siRNA results in reduced cell proliferation. Human MCF7 mammary carcinoma cells (FIG. 4A) and normal human keratinocytes (HaCat cell line) (FIG. 4B) in which Sdc1 expression is silenced show dramatically reduced proliferation over the ensuing 72 hr. The block to proliferation becomes most dramatic during 48-72 hr after siRNA treatment, which is the period of time when silencing of Sdc1 by the siRNA is most effective (FIG. 4A-B).

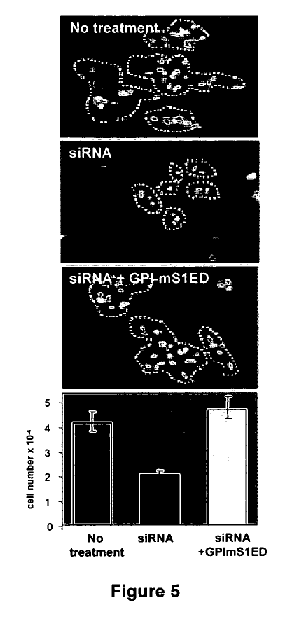

Rescue of proliferation in the siRNA-treated cells was seen when the cells are transfected with mouse Sdc1 (not shown), or with mouse Sdc1 extracellular domain alone (GPI-mS1ED, FIG. 5); in the latter case, the extracellular domain is anchored to the membrane via a glycosylphosphatidylinositol (GPI) tail that inserts into the outer leaflet of the plasma membrane. The fact that the expression of this Sdc1 mutant rescues the proliferative phenotype of the cells is consistent with the block to proliferation seen in the presence of antibodies directed against the Sdc1 extracellular domain, and suggests that a determinant in this extracellular protein domain is necessary and sufficient for the regulatory effect.

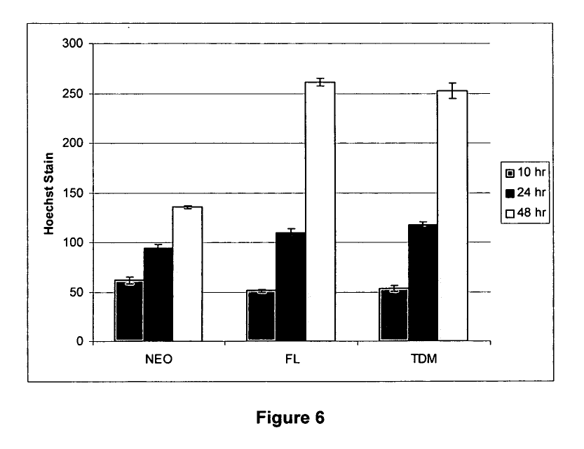

Rescue of proliferation is also seen when the cells are transfected with a mouse Sdc1 mutant lacking its heparan sulfate chains (TDM mutant, FIG. 6). This is shown in MDA-MB-231 human mammary carcinoma cells in which the endogenous human SDc1 expression is silenced with human-specific siRNA and is replaced either with mouse Sdc1 or with mouse Sdc1 in which the three attachement sites for heparan sulfate chains have been mutated (triple-deletion mutant or TDM). Transfection with the mouse Sdc1 rescues proliferation compared to parental cells and the TDM mutant also rescues to the same extent as the native mouse Sdc1. This provides further evidence that the active site in the Sdc1 is a protein epitope in the extracellular domain of the Sdc1 protein.

As a test for cell type specificity, the block to proliferation seen when treating cells with Sdc1-specific antibody was tested on SH-SY5Y human neuroblastoma cells. Cells were placed in Matrigel™ together with no antibody, 200 μg/ml nonspecific rabbit antibody, or 200 μg/ml rabbit anti-Sdc1 antibody, which was identical to the treatments used for the human mammary carcinoma cells. The SH-SY5Y cells recapitulated the response seen with the mammary cells, namely, small colonies of cells were visible after three day culture in the absence of antibody, or in the presence of nonspecific rabbit IgG, but only single cells that failed to proliferate to form colonies were seen in the Sdc1 antibody.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,554,101
U.S. Pat. No. 5,446,128
U.S. Pat. No. 5,710,245
U.S. Pat. No. 5,840,833
U.S. Pat. No. 5,859,184
U.S. Pat. No. 5,440,013
U.S. Pat. No. 5,618,914
U.S. Pat. No. 5,670,155
U.S. Pat. No. 5,475,085
U.S. Pat. No. 5,929,237
U.S. Pat. No. 5,672,681
U.S. Pat. No. 5,674,976
Adams et al., J. Cell Biol., 152:1169-1182, 2000.
Alexander et al., Nat. Genet., 25:329-332, 2000.
Antibodies: A Laboratory Manual, Cold Spring Harbor Laboratory, Cold Spring Harbor Press, Cold Spring Harbor, N.Y., 1988.
Anttonen et al., Br. J. Cancer, 79:558-564, 1999.
Bachelder et al., J. Biol. Chem., 274:20733-20737, 1999.
Baichwal and Sugden, In: Gene Transfer, Kucherlapati (Ed.), NY, Plenum Press, 117-148, 1986.
Bakke et al., Cell, 63(4):707-716, 1990.
Barany and Merrifield, In: The Peptides, Gross and Meienhofer (Eds.), Academic Press, NY, 1-284, 1979.
Barbareschi et al., Cancer, 98:474-483, 2003.
Bayer-Garner et al., J. Cutan. Pathol., 28:135-139, 2001.
Beauvais and Rapraeger, Exp. Cell Res., 286:219-232, 2003.
Beauvais et al., J. Cell Biol., 167:171-181, 2004
Becker-Hapak et al., Methods, 24:247-256, 2001.
Benvenisty and Neshif, Proc. Natl. Acad. Sci. USA, 83(24):9551-9555, 1986.
Bemfield et al., Annu. Rev. Biochem., 68:729-777, 1999.
Brassard et al., Exp. Cell Res., 251:33-45, 1999.
Brooks et al., Cell, 1994 Dec 30;79(7):1157-1164, 1994.
Burbach et al., Matrix Biol., 22:163-177, 2003.
Capaldi et al., Biochem. Biophys. Res. Comm., 74(2):425-433, 1977.
Chen and Okayama, Mol. Cell Biol., 7(8):2745-2752, 1987.
Coffin, In: Virology, Fields et al. (Eds.), Raven Press, NY, 1437-1500, 1990.
Cohen et al., J. Cell Biol., 142:129-138, 1998.
Conejo et al., Int. J Cancer, 88:12-20, 2000.
Coupar et al., Gene, 68:1-10, 1988.
Crescimanno et al., J. Pathol., 189:600-608, 1999.
Denicourt and Dowdy, Trends Pharmacol. Sci., 24:216-218, 2003.
Domanico et al., Mol. Biol. Cell, 8:2253-2266, 1997.
Dubensky et al., Proc. Natl. Acad. Sci. USA, 81:7529-7533, 1984.
Ethell et al., J. Cell Biol., 151:53-68, 2000.
Fechheimer, et al., Proc Natl. Acad. Sci. USA, 84:8463-8467, 1987.
Fraley et al., Proc. Natl. Acad. Sci. USA, 76:3348-3352, 1979.
Fujiya et al., Jpn J. Cancer Res., 92:1074-1081, 2001.
Gao et al., J. Cell Physiol., 184:373-379, 2000.
Ghosh and Bachhawat, In: Liver Diseases, Targeted Diagnosis and Therapy Using Specific Receptors and Ligands, Wu et al. (Eds.), Marcel Dekker, NY, 87-104, 1991.
Gopal, Mol. Cell Biol., 5:1188-1190, 1985.
Graham and Van Der Eb, Virology, 52:456-467, 1973.
Granes et al., J. Cell Sci., 113:1267-1276, 2000.
Grootjans et al., Proc. Natl. Acad Sci. USA, 94:13683-13688, 1997.
Harland and Weintraub, J. Cell Biol., 101(3):1094-1099, 1985.
Harlow et al., Mol. Cell Biol., 5(7): 1601-1610, 1985.
Hirabayashi et al., Tumour Biol., 19:454-463, 1998.
Hsueh et al., J. Cell Biol., 142:139-151, 1998.
Hsueh et al., J. Neurosci., 21(11):3764-7370, 2001.
Iba et al., J. Cell Biol., 149:1143-1156, 2000.
Inki and Jalkanen, Ann. Med., 28:63-67, 1996.
Inki et al., Br. J. Cancer, 70:319-323, 1994.
Inki et al., J. Pathol., 172:349-355, 1994.
Johannesson et al. J. Med. Chem., Nov. 4, 1999;42(22):4524-37, 1999.
Johnson et al., In: Biotechnology And Pharmacy, Pezzuto et al. (Eds.), Chapman and Hall, NY, 1993.
Kato et al., Mol. Biol. Cell, 6:559-576, 1995.
Kim et al, Mol. Biol. Cell, 5(7):797-798, 1994.
Klatka, Eur. Arch. Otorhinolaryngol., 259:11, 2002.
Kubota et al., Biochem. Biophys. Res. Commun., 162(3):963-970, 1989.
Kumar-Singh et al., J. Pathol., 186:300-305, 1998.
Kuzuyu et al., Exp. Cell Res., 248(2):498-508, 1999.
Kyte and Doolittle, J. Mol. Biol., 157(1):105-132, 1982.
Lanford et al., Cell, 46(4):575-582, 1986.
Lebakken et al., J. Cell Biol., 132:1209-1221, 1996.
Leppa et al., Cell Regul., 2:1-11, 1991.
Leppaet al., J. Cell Sci., 109:1393-1403, 1996.
Leppa et al., Proc. Natl. Acad. Sci USA, 89:932-936, 1992.
Letourneur et al., Cell, 69(7):1143-1157, 1992.
Levy et al., Br. J. Cancer, 74:423-431, 1996.
Levy et al., Bull. Cancer, 84:235-237, 1997.
Lewis et al., Proc. Natl. Acad. Sci. USA, 99:3627-3632, 2002.
Liu et al., J. Biol. Chem., 273(35):22825-22832, 1998.
Mali et al., J. Biol. Chem., 269:27795-27798, 1994.
Matsumoto et al., Int. J. Cancer, 74:482-491, 1997.
McFall and Rapraeger, J. Biol. Chem., 272:12901-12904, 1997.
McFall and Rapraeger, J. Biol. Chem., 273:28270-28276, 1998.
McQuade and Rapraeger, J. Biol. Chem., 278(47):46607-46615, 2003.
McQuade et al., J. Cell Sci., 119:2445-2456, 2006.
Merrifield, Science, 232(4748):341-347, 1986.
Mundhenke et al., Am. J. Pathol., 160:185-194, 2002.
Munro et al., Cell, 48(5):899-907, 1987.
Nackaerts et al., Int. J. Cancer, 74:335-345, 1997.
Nakanishi et al., Int. J. Cancer, 80:527-532, 1999.
Nicolau and Sene, Biochim. Biophys. Acta, 721:185-190, 1982.
Numa et al., Int. J Oncol., 20:39-43, 2002.
Ohtake et al., Br. J. Cancer, 81:393-403, 1999.
Park et al., J. Biol. Chem., 277:29730-29736, 2002.
PCT Appln. WO 84/03564
Potter et al., Proc. Natl. Acad. Sci. USA, 81:7161-7165, 1984.
Pulkkinen et al., Acta Otolaryngol., 117:312-315, 1997.
Radler et al., Science, 275:810-814, 1997.
Rapraeger and Ott, Curr. Opin. Cell Biol., 10(5):620-628, 1998.
Rapraeger et al., J. Cell Biol., 103:2683-2696, 1986.
Remington's Pharmaceutical Sciences, 18th Ed. Mack Printing Company, 1990.
Ridgeway, In: Vectors: A survey of molecular cloning vectors and their uses, Rodriguez and Denhardt (Eds.), Stoneham:Butterworth, 467-492, 1988.
Rintala et al., Gynecol. Oncol., 75:372-378, 1999.
Rippe et al., Mol. Cell Biol., 10:689-695, 1990.
Sanderson et al., Proc. Natl. Acad. Sci. USA, 95:9562-9566, 1988.
Sanderson, Ann. Hematol., 81:12, 2002.
Sanderson, Semin. Cell Dev. Biol., 12(2):89-98, 2001.
Sasisekharan et al., Nat. Rev. Cancer, 2:521-528, 2002.
Schwarze et al., Trends Cell Biol., 10:290-295, 2000.
Siomi et al., Cell, 55(2):197-209, 1988.
Song et al., Proc. Natl. Acad. Sci. USA, 96:1100-1105, 1999.
Soukka et al., J. Oral. Pathol. Med., 29:308-313, 2000.
Stanley et al., Am. J. Clin. Pathol., 112:377-383, 1999.
Stanton et al., Proc. Natl. Acad. Sci. USA, 83(6):1772-1776, 1986.
Stewart and Young, In: Solid Phase Peptide Synthesis, 2d. ed., Pierce Chemical Co., 1984.
Stupack et al., J. Cell Biol., 155:459-470, 2001.
Sun et al., Int. J. Dev. Biol., 42(5):733-736, 1998.
Tam et al., J. Am. Chem. Soc., 105:6442, 1983.
Thodeti et al., J. Biol. Chem., 278:9576-9584, 2003.
Tur-Kaspa et al., Mol. Cell Biol., 6:716-718, 1986.
U.S. Pat. No. 5,482,856
Vita et al., Biopolymers, 47:93-100, 1998.
Weisshoff et al., Eur. J. Biochem., 259(3):776-788, 1999.
Wender et al., Proc. Natl. Acad. Sci. USA, 97:13003-13008, 2000.
Wiksten et al., Int. J. Cancer, 95(1):1-6, 2001.
Wu and Wu, Biochemistry, 27:887-892, 1988.
Wu and Wu, J. Biol. Chem., 262:4429-4432, 1987.
Yang et al., Proc. Natl. Acad. Sci. USA, 87:9568-9572, 1990.
Yellon et al., Cardiovasc Res., 26(10):983-987, 1992.

What is claimed is:

1. A method of inhibiting a cancer cell comprising contacting said cancer cell with a syndecan 1 ectodomain peptide, wherein said peptide lacks heparan sulfate residues.

2. The method of claim 1, wherein said peptide is between 5 and 100 residues in length.

3. The method of claim 2, wherein said peptide is between 15 and 50 residues in length.

4. The method of claim 3, wherein said peptide is between 25 and 40 residues in length.

5. The method of claim 1, wherein said cancer cell is an epithelial cancer cell or a carcinoma.

6. The method of claim 1, wherein inhibiting comprises one or more of inhibiting cancer cell growth, inhibiting cancer cell proliferation, or inhibiting cancer cell differentiation.

7. The method of claim 1, wherein said cancer cell is a drug-resistant cancer cell or a metastatic cancer cell.

8. The method of claim 1, further comprising contacting said cancer cell with a second anti-cancer treatment.

9. The method of claim 8, wherein said second anti-cancer treatment comprises radiation, chemotherapy, hormonal therapy, gene therapy or immunotherapy.

10. The method of claim 1, further comprising contacting said cancer cell a second time with said peptide.

11. A method of treating a subject with cancer comprising administering to said subject a syndecan 1 ectodomain peptide, wherein said peptide lacks heparan sulfate residues.

12. The method of claim 11, wherein said peptide is between 5 and 100 residues in length.

13. The method of claim 12, wherein said peptide is between 15 and 50 residues in length.

14. The method of claim 13, wherein said peptide is between 25 and 40 residues in length.

15. The method of claim 1 1, wherein said cancer is an epithelial cancer or a carcinoma.

16. The method of claim 11, wherein inhibiting comprises one or more of inhibiting cancer cell growth, inhibiting cancer cell proliferation, inhibiting cancer cell differentiation, inhibiting cancer progression, inhibiting cancer metastasis, or inhibiting cancer recurrence.

17. The method of claim 11, wherein said cancer cell is a drug-resistant cancer cell or a metastatic cancer cell.

18. The method of claim 11, further comprising contacting said subject with a second anti-cancer treatment.

19. The method of claim 18, wherein said second anti-cancer treatment comprises radiation, chemotherapy, hormonal therapy, gene therapy, immunotherapy or surgery.

20. The method of claim 11, further comprising administering said peptide to said subject a second time.

21. A method of treating a subject with cancer comprising contacting said cancer cell with an agent that binds syndecan 1.

22. The method of claim 21, wherein said agent is a peptide, peptidomimetic or polypeptide.

23. The method of claim 22, wherein said polypeptide is an antibody or antibody fragment.

24. The method of claim 21, wherein said agent is an organopharmaceutical.

25. The method of claim 21, wherein said cancer is an epithelia cancer cell or a carcinoma.

26. The method of claim 21, wherein inhibiting comprises one or more of inhibiting cancer cell growth, inhibiting cancer cell proliferation, inhibiting cancer cell differentiation, inhibiting cancer progression, inhibiting cancer metastasis, or inhibiting cancer recurrence.

27. The method of claim 21, wherein said cancer cell is a drug-resistant cancer cell or a metastatic cancer cell.

28. The method of claim 21, further comprising contacting said subject with a second anti-cancer treatment.

29. The method of claim 28, wherein said second anti-cancer treatment comprises radiation, chemotherapy, hormonal therapy, gene therapy, immunotherapy or surgery.

30. The method of claim 21, further comprising administering said agent to said subject a second time.

31. A method of screening for a candidate anti-cancer agent comprising:
(a) providing a syndecan 1 ectodomain;
(b) contacting said syndecan 1 ectodomain with test compound;
(c) assessing binding of said test compound to said syndecan 1 ectodomain;
wherein an agent that binds to said syndecan 1 ectodomain is a candidate anti-cancer agent.

32. The method of claim 31, wherein said syndecan 1 ectodomain is comprised within full length syndecan 1.

33. The method of claim 32, wherein full length syndecan 1 is embedded in a membrane.

34. The method of claim 33, wherein full length syndecan 1 is located in an intact cell membrane.

35. The method of claim 34, wherein said cell membrane is dervied from a cell recombinantly engineered to express syndecan 1.

36. The method of claim 31, wherein syndecan 1 ectodomain is fixed to a support.

37. The method of claim 36, wherein said support is a stick, a well, a bead or a column matrix.

38. The method of claim 31, wherein binding is detected by mass spectrometry, cell sorting, fluorescence, sedimentation, electrical current, FRET, chromatography, and solid phase adhesion.

39. The method of claim 31, further comprising assessing the effect of said test compound on cancer cell growth.

40. The method of claim 31, wherein said test compound is a peptide, a polypeptide, a peptidomimetic, a nucleic acid, a carbohydrate, a lipid, or a organopharmaceutical.

41. A method of screening a syndecan 1 ectodomain peptide for anti-cancer activity comprising:
(a) providing a syndecan 1 ectodomain peptide;
(b) contacting said peptide with a cancer cell; and
(c) assessing the effect of said peptide on said cancer cell growth or viability,
wherein a peptide that inhibits cancer cell growth or viability has anti-cancer activity.


SEQUENCE LISTING (4 sequences)

SEQ ID NO: 1 - protein, 310 aa, Homo sapiens
MRRAALWLWLCALALSLQPALPQIVATNLPPEDQDGSGDDSDNFSGSGAGALQDITLSQQTPSTWKDTQLLTAIPTSPEPTGLEATAASTSTLPAGEGPKEGEAVVLPEVEPGLTAREQEATPRPRETTQLPTTHQASTTTATTAQEPATSHPHRDMQPGHHETSTPAGPSQADLHTPHTEDGGPSATERAAEDGASSQLPAAEGSGEQDFTFETSGENTAVVAVEPDRRNQSPVDQGATGASQGLLDRKEVLGGVIAGGLVGLIFAVCLVGFMLYRMKKKDEGSYSLEEPKQANGGAYQKPTKQEEFYA

SEQ ID NO: 2 - DNA, 930 bp, Homo sapiens
atgaggcgcgcggcgctctggctctggctgtgcgcgctggcgctgagcctgcagccggccctgccgcaaattgtggctactaatttgccccctgaagatcaagatggctctggggatgactctgacaacttctccggctcaggtgcaggtgctttgcaagatatcaccttgtcacagcagaccccctccacttggaaggacacgcagctcctgacggctattcccacgtctccagaacccaccggcctggaggctacagctgcctccacctccaccctgccggctggagaggggcccaaggagggagaggctgtagtcctgccagaagtggagcctggcctcaccgcccgggagcaggaggccaccccccgacccagggagaccacacagctcccgaccactcatcaggcctcaacgaccacagccaccacggcccaggagcccgccacctcccacccccacagggacatgcagcctggccaccatgagacctcaacccctgcaggacccagccaagctgaccttcacactccccacacagaggatggaggtccttctgccaccgagagggctgctgaggatggagcctccagtcagctcccagcagcagagggctctggggagcaggacttcacctttgaaacctcgggggagaatacggctgtagtggccgtggagcctgaccgccggaaccagtccccagtggatcagggggccacgggggcctcacagggcctcctggacaggaaagaggtgctgggaggggtcattgccggaggcctcgtggggctcatctttgctgtgtgcctggtgggtttcatgctgtaccgcatgaagaagaaggacgaaggcagctactccttggaggagccgaaacaagccaacggcggggcctaccagaagcccaccaaacaggaggaattctatgcc

SEQ ID NO: 3 - protein, 234 aa, Homo sapiens
QPALPQIVATNLPPEDQDGSGDDSDNFSGSGAGALQDITLSQQTPSTWKDTQLLTAIPTSPEPTGLEATAASTSTLPAGEGPKEGEAVVLPEVEPGLTAREQEATPRPRETTQLPTTHQASTTTATTAQEPATSHPHRDMQPGHHETSTPAGPSQADLHTPHTEDGGPSATERAAEDGASSQLPAAEGSGEQDFTFETSGENTAVVAVEPDRRNQSPVDQGATGASQGLLDRKE

SEQ ID NO: 4 - DNA, 701 bp, Homo sapiens
agccggccctgccgcaaattgtggctactaatttgccccctgaagatcaagatggctctggggatgactctgacaacttctccggctcaggtgcaggtgctttgcaagatatcaccttgtcacagcagaccccctccacttggaaggacacgcagctcctgacggctattcccacgtctccagaacccaccggcctggaggctacagctgcctccacctccaccctgccggctggagaggggcccaaggagggagaggctgtagtcctgccagaagtggagcctggcctcaccgcccgggagcaggaggccaccccccgacccagggagaccacacagctcccgaccactcatcaggcctcaacgaccacagccaccacggcccaggagcccgccacctcccacccccacagggacatgcagcctggccaccatgagacctcaacccctgcaggacccagccaagctgaccttcacactccccacacagaggatggaggtccttctgccaccgagagggctgctgaggatggagcctccagtcagctcccagcagcagagggctctggggagcaggacttcacctttgaaacctcgggggagaatacggctgtagtggccgtggagcctgaccgccggaaccagtccccagtggatcagggggccacgggggcctcacagggcctcctggacaggaaagag